(19) United States
(12) Patent Application Publication
(10) Pub. No.: US 2022/0079820 A1
(43) Pub. Date: Mar. 17, 2022

(54) Butterfly Elastic Band Stretch Disposal Underwear with Bladder Protection

(71) Applicant: Nikola T. Warren, New Kensington, PA (US)

(72) Inventor: Nikola T. Warren, New Kensington, PA (US)

(21) Appl. No.: 17/234,078

(22) Filed: Apr. 19, 2021

Related U.S. Application Data
(60) Provisional application No. 62/958,263, filed on Apr. 20, 2020.

(51) Int. Cl. A61F 13/56; A61F 13/47

(52) U.S. Cl. CPC A61F 13/5605; A61F 2013/15146; A61F 13/47

(57) ABSTRACT

This invention is designed to allow women to wear disposable strap underwear with bladder pad protection that fits and stretches around the leg rather than the thigh. The stretchable elastic bands claim without size restriction allows the bands to be used in unisex to secure pads, underwear, or pants.

The Butterfly Elastic Band Stretch Disposal Underwear with Bladder protection is designed to wear pad as disposable underwear by using a button elastic strap to fasten comfortably to the four corners of the rectangular pad. The elastic strap with buttons stretches and fits securely around the upper shape of the leg. The elastic strap with buttons is stretchable and can be used to secure a disposable pad, underwear, or pants. The elastic strap can either be used with buttons for reuse or bonded to the pad for disposal. The pad used with the elastic band stretch disposal underwear can either have an insert or slit in four corners for the button to fasten or the strap can be bonded to the pad.

### DESCRIPTION OF DRAWING

FIG. 1

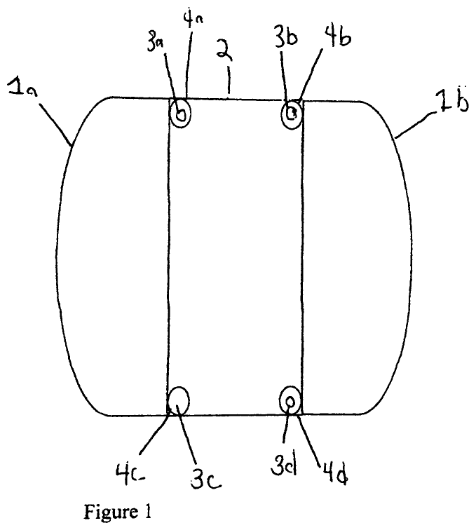

1a. and 1b. The stretchable elastic strap is attached to 4a, 4b, 4c, 4d, which are buttons and inserted into 3a, 3b, 3c, 3d, hole inserts in the four corners of 2. rectangular pad.

FIG. 2

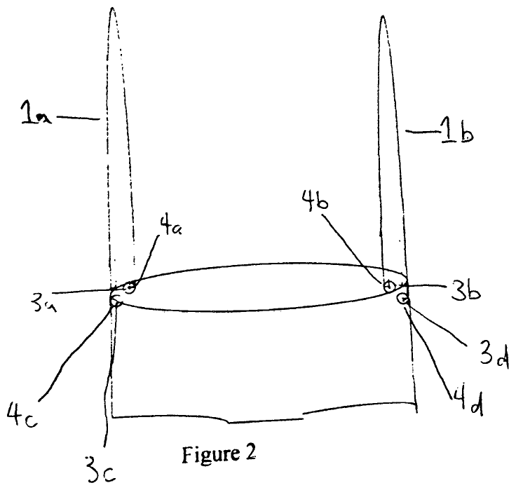

1a and 1b. The stretchable elastic strap is attached to 4a, 4b, 4c, 4d, which are buttons and inserted into 3a, 3b, 3c, 3d, hole inserts in the four corners of underwear or pants.

FIG. 3

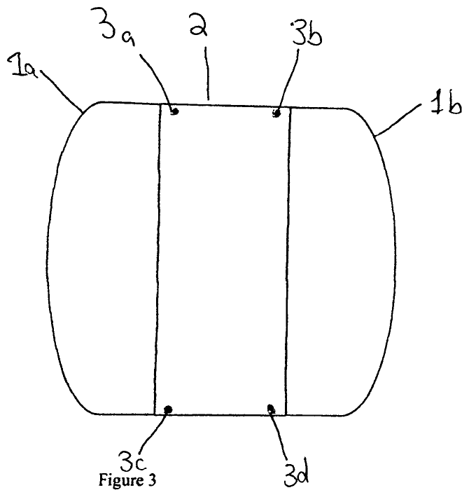

1a and 1b. The stretchable elastic strap is bonded to 3a, 3b, 3c, 3d, located in the four corners of the 2. rectangular pad

What is claimed is for Ser. No. 17/234,078:

1. Absorbent rectangle pad with hole inserts in four corners.

2. Absorbent rectangle pad with bonded strap attached to four corners.

3. Stretchable Elastic Strap with Buttons attached or Bonded to each end.

4. (canceled)
